(19) United States
(12) Patent Application Publication
(10) Pub. No.: US 2020/0337868 A1
(43) Pub. Date: Oct. 29, 2020

(54) SYSTEM, METHOD AND APPARATUS OF COIL AND ROD CONSTRUCTION COMPRISING MECHANISMS SUCH AS THE COIL JOINT, COIL BODY AND COIL JOINT SPINE

(71) Applicant: Jose HERMINIO Callas, Seattle, WA (US)

(72) Inventor: Jose HERMINIO Callas, Seattle, WA (US)

(21) Appl. No.: 16/848,248

(22) Filed: Apr. 14, 2020

Related U.S. Application Data
(60) Provisional application No. 62/838,091, filed on Apr. 24, 2019.

(51) Int. Cl. A61F 2/68; A61F 2/58

(52) U.S. Cl. CPC A61F 2/68; A61F 2002/741; A61F 2/586

(57) ABSTRACT

A system and method of construction based on rods and coils, that includes multiple parts comprising: distinct apparatuses, one or more coil joints, a coil body, and one or more coil spines; wherein any combination of the above parts can result in a variety of distinct structures; wherein the coil joint has 360 degree rotation; wherein the rotation can be limited to simulate other types of joints; wherein this coil joint can be combined with other joint styles to create hybrid styles; wherein there are 2 bias actions, one is an extending action and the other is a contracting action; wherein the action of the coil results in a more life-like motion than not using the coil; wherein the coil body has a flexible shape that can be locked into place; wherein the coil body has a high strength to weight ratio, which leads to high dimensional stability; wherein there is also hollow space for storage and transport of a package inside of the coil body; wherein the coil spine simulates the spines of living beings; wherein the coil spine is a flexible yet robust structure.

A system and method of construction based on rods and coils that can create a mechanical hand; wherein the coil joints function as the equivalent to human joints; wherein there are rods that function as the equivalent to human bones; wherein the coil joints create a dampening effect that simulates the dampening effect in human hands; wherein there are coil bodies of the hand, that are used for transport and storage; wherein the fingers of the artificial hand can be replaced with tools that are of a similar size and not excessive weight; wherein the coil joints can be modified to simulate various human joints; where there is a bias in all coils and coil joints; wherein the bias of the coil joints increases the ability of the artificial hand to grasp; wherein the bias of the coil joint functions as tendon tension; wherein the bias of all the coils results in more life-like motion; wherein movement of the finger can be performed with a single wire opposing the bias of the coil; wherein the artificial hand can have autonomous motion by utilizing an actuator system.

### FIELD OF THE INVENTION

The field of the invention is rod and coil construction systems.

These are systems in which the basic structure of the rod and coil are combined to obtain distinct beneficial structural characteristics.

### BACKGROUND

The background description includes information that may be useful in understanding the present invention. It is not an admission that any of the information provided herein is prior art or relevant to the presently claimed invention, or that any publication specifically or implicitly referenced is prior art.

All publications identified herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

In current industry usage, there is a use of springs and spring extensions. There are package bodies that provide extra strength, and there are package bodies that provide shock absorption or shock transfer, as well as protection from shock and vibration.

Artificial hands typically use the hinge joint. Current artificial hands are directed to the prosthetic market.

In terms of existing joints, there is the ball and socket joint, which has no memory, and no shock resistance. There is the swivel joint, which has no memory, and no shock resistance. There is the saddle joint, which has no memory and no shock resistance.

In terms of existing springs and coils, there is the extension version, which is not a connection between solid bodies. There is the compression version, which also is not a connection between solid bodies.

There is the bobble head doll, which is not a system of construction, and in which no weight bearing is considered.

In terms of existing spring or coil tubes or bodies, there is the top hat version, which has limited usage and no system for general use. There is the flexible tubing or duct version, but it has no way to maintain the desired shape. There is the underground tubes or tunneling version, which lacks flexibility. Lastly there are lamps, which lacks the ability to change and maintain shape.

In terms of existing artificial hands, there are numerous problems:
it is hard to get new parts
They are limited to the task of grasping
Fluid delivery not considered
There is no light delivery
There is no consideration of fingertips as distinct tools
Only the standard hinge joints are used in existing artificial hands

In terms of Existing Packages: Cardboard does not stack well, does not protect well from crushing, vibration, theft, and cannot be used as a strut, lacks rigidity. A crate does not readily change shape. A custom package cannot be used for objects of varying sizes. A collapsible tube lacks the built-in ability to maintain shape.

The inventions in U.S. Pat. Nos. 1,409,513A and 2,542,316A use springs.

The device in U.S. Pat. No. 1,409,513A would not actually function as intended.

The problems described above with all of these joints, springs, coils, artificial hands and packages requires a solution.

### SUMMARY

The present invention utilizes the characteristics of the coil in combination with rods to implement distinct construction systems. The coil and rod structure consists mainly of three distinct mechanisms, the Coil Joint, Coil Body, and Coil Rod Spine. A Coil Joint, a coil to connect solid bodies, primarily rods. A Coil Body, a coil woven through one or more rods to create a hollow body. A Coil Rod Spine, a series of Coil Joints and rods to build flexible yet robust structures.

Some of the distinct characteristics of the coil capitalized upon are:

joints with bias, compression, and extension, the characteristics of a coil.

One embodiment of this construction system is an artificial hand in which Coil Joints simulate the human joints.

Another embodiment of this construction system is a package, in which coil bodies with coil Rod spines function as a package.

Differences between the prior art and the present invention

The prior art does not make use of the space inside the hand for anything except the motion of the hand itself. In contrast, the present invention does utilize space inside the hand for action beyond just the motion of the hand itself.

The artificial bands in the present invention have many uses and could be brought into markets as a better solution. In particular, I am thinking of the Cleaning Hand.

The prior art does not include any packaging that uses the helix shape.

The prior art does not include any packaging that locks in its changed shape.

U.S. Pat. Nos. 1,409,513A and 2,542,316A use springs but neither uses a Spring Joint.

It appears that U.S. Pat. No. 1,409,513A would not actually function as intended.

U.S. Pat. No. 2,542,316A uses an extension spring as shown in FIG. 3 of U.S. Pat. No. 2,542,316A, but this use is distinct from the Spring Joint in the present invention.

### BRIEF DESCRIPTION OF THE DRAWINGS

The following drawings are only part of the embodiments of the present invention, rather than all embodiments. A person skilled in the art may achieve other drawings based on the following ones without any creative efforts.

The accompanying drawings, which are included to provide a further understanding of the disclosed subject matter, are incorporated in and constitute a part of this specification. The drawings also illustrate implementations of the disclosed subject matter and together with the detailed description serve to explain the principles of implementations of the disclosed subject matter. No attempt is made to show structural details in more detail than may be necessary for a fundamental understanding of the disclosed subject matter and various ways in which it may be practiced.

FIG. 1A shows two details of the hand joint of one embodiment of the Rod and Coil Construction System.

FIG. 1B shows two details of the hand joint of one embodiment of the Rod and Coil Construction System.

FIG. 2A shows a layout of the joints and how they would be incorporated into the hand of one embodiment of the Rod and Coil Construction System.

FIG. 2B shows a layout of the joints and how they would be incorporated into the hand of one embodiment of the Rod and Coil Construction System.

FIG. 2C shows a layout of the joints and how they would be incorporated into the hand of one embodiment of the Rod and Coil Construction System.

FIG. 3 shows a general image of the package of one embodiment of the Rod and Coil Construction System.

FIG. 4A shapes the package can take and two after delivery uses depicted of one embodiment of the Rod and Coil Construction System.

FIG. 4B shapes the package can take and two after delivery uses depicted of one embodiment of the Rod and Coil Construction System.

FIG. 4C shapes the package can take and two after delivery uses depicted of one embodiment of the Rod and Coil Construction System.

FIG. 4D shapes the package can take and two after delivery uses depicted of one embodiment of the Rod and Coil Construction System.

FIG. 4E shapes the package can take and two after delivery uses depicted of one embodiment of the Rod and Coil Construction System.

FIG. 4F shapes the package can take and two after delivery uses depicted of one embodiment of the Rod and Coil Construction System.

FIG. 5 displays the artificial hand made through the rod and coil construction system

FIG. 6 displays the artificial hand made through the rod and coil construction system, with additional details.

FIG. 7 displays 1 connection in the artificial hand made through the rod and coil construction system

FIG. 8 displays 1 connection in the artificial hand made through the rod and coil construction system, with additional details.

FIG. 9 displays a cylindrical design, in which a coil goes through rods from the bottom to top of an otherwise open-sided cylinder, which has a removable top.

FIG. 10 displays a cylindrical design, in which a coil goes through rods from the bottom to top of an otherwise open-sided cylinder, which has a removable top. This version differs from FIG. 9 because it has additional details.

FIG. 11 displays a cylindrical design, in which a coil goes through rods from the bottom to top of an otherwise open-sided cylinder, and at a certain height there are coil joints used to secure the connection to the rods, as opposed to the coil going through the rods, which it does everywhere else vertically on the rod.

FIG. 12 displays a cylindrical design, in which a coil goes through rods from the bottom to top of an otherwise open-sided cylinder, and at a certain height there are coil joints used to secure the connection to the rods, as opposed to the coil going through the rods, which it does everywhere else vertically on the rod. This package differs from FIG. 11 because it contains more details.

FIG. 13 displays a cylindrical design, in which a coil goes through rods from the bottom to top of an otherwise open-sided cylinder, which has a removable top. At a certain height there are coil joints used to secure the connection to the rods, as opposed to the coil going through the rods, which it does everywhere else vertically on the rod.

FIG. 14 displays a cylindrical design, in which a coil goes through rods from the bottom to top of an otherwise open-sided cylinder, which has a removable top. At a certain height there are coil Joints used to secure the connection to the rods, as opposed to the coil going through the rods, which it does everywhere else vertically on the rod. This package differs from FIG. 13 because it contains more details.

FIG. 15 displays the flat almost circular bottom of the package.

FIG. 16 displays the flat almost circular bottom of the package, with additional details.

FIG. 17 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod.

FIG. 18 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod. This package differs from FIG. 17 because it contains more details.

FIG. 19 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod.

FIG. 20 displays a cylindrical design, in which a coil goes around rods from the bottom to top of an otherwise open-sided cylinder, which has a removable top. At a certain height there are coil joints used to secure the connection to the rods, as opposed to the coil going through the rods which it does everywhere else vertically on the rod. This package differs from FIG. 19 because it contains more details.

FIG. 21 displays the 2 stages of the package, one in which the package is expanded, and the other in which the package is contracted. There is a total height difference, as well as a reduction in distance between the height of each round of coil, as well as a reduction in the height of the coil joints.

FIG. 22 displays the 2 stages of the package, one in which the package is expanded, and the other in which the package is contracted. There is a total height difference, as well as a reduction in distance between the height of each round of coil, as well as a reduction in the height of the coil joints. This figure differs from FIG. 21 because it has additional details.

FIG. 23 displays the package from a top viewpoint, and with a full cover, such that there is no opening between the coils or the rods to the outside.

FIG. 24 displays a cylindrical design, in which a coil goes through rods from the bottom to top of an otherwise open-sided cylinder, which has a removable top, and in this case the top is closed. The view is from about 45 degrees up, between horizontal and vertical.

FIG. 25 displays a cylindrical design, in which a coil goes through rods from the bottom to top of an otherwise open-sided cylinder, which has a removable top, and in this case the top is closed. The view is almost horizontal.

FIG. 26 displays a cylindrical design, in which a coil goes through rods from the bottom to top of an otherwise open-sided cylinder, which has a removable top, and in this case the top is closed. The view is from below the horizontal plane.

FIG. 27 displays a close up of the coil joint, in which you can see coils above and below the joint going through the rod, as well as the coil joint curled around the rod. This version is when the package is compressed.

FIG. 28 displays a close up of the coil joint, and in this version the package is open such that a gap is created between the top part of the rod and the bottom part of the rod. The coil joint is able to retain the connection between the top and bottom parts of the rod. The flexibility of the coil joint is what allows the package to expand and compress.

FIG. 29 displays the flat almost circular top of the package, with the lid closed, where the lid is a little inside from the edge.

FIG. 30 displays an artificial hand with rods and coil joints.

FIG. 31 displays an artificial hand with rods and coil joints. This package differs from FIG. 30 because it contains more details.

FIG. 32 displays an artificial hand with rods and coil joints.

FIG. 33 displays an artificial hand with rods and coil joints. This package differs from FIG. 32 because it contains more details.

FIG. 34 displays an artificial hand with rods and coil joints.

FIG. 35 displays an artificial hand with rods and coil joints. This package differs from FIG. 34 because it contains more details.

### DETAILED DESCRIPTION

A system and method are disclosed wherein the main apparatus that characterize this type of construction consist of coils and rods.

The combined usage of these rods and coils, utilize the characteristics of the coil to develop apparatus with distinct qualities.

Distinct Apparatuses:

1. Coil Joint

A coil is used to connect two solid bodies. These joints have a full 360-degree rotation. Coil Joints spring back to their original positions. Coil Joints can function in place of currently existing joints and will take its place alongside existing standard joints to be another standardly used type of joint in any number of applications.

Along with the rotation,

there is an extending and contracting motion as well. Restricted motion is achieved through various techniques.

2. Coil Body

A coil is threaded through one or more rods while retaining its shape and reinforced by these rods. The threading of the coil can be adjusted to create bodies with various shapes. The Coil Body has distinct vibrational properties that enable shock absorption and or transfer.

3. Coil Joint Spines

A series of Coil Joints connected by rods form a strong and flexible structure. These Coil Joint Spines function as a kind of a strut for a given structure, providing support yet flexibility for the shape.

Specific Qualities of these techniques are:
High strength-to-weight ratio,
High dimensional stability Coil Body
Collapsible stretchable, and malleable Coil/Coil Spine bodies
Hollow coil body
Bias, the dampening and memory effects of the construction result in a more lifelike motion and variable size.
Full 360-degree rotation of Coil Joint
Vibrational qualities for shock/vibration absorption and transfer

Two applications of Rod and (Coil construction are:
Mechanical Hand
Package

Mechanical Hand

An embodiment of the present invention is as a mechanical hand. The Coil Joint functions as the human joint. The rod functions as the equivalent to human bone. The characteristics of the Coil Joints give the motion of the hand a realistic appearance. The dampening effect of the coils simulates the human bodies' dampening. Coil bias provides the mechanical hand with lifelike qualities of movement. Actuators and pulley systems can be attached to the rods to be used to move and control the hand. Coil Bodies form the shape of the hand itself with hollow bodies for transport or storage.

Furthermore, the Coil Joints' bias can be used as tendon tension. This enables finger action to be accomplished with a single ‘tendon’ wire.

Industry Usage of the Mechanical Hand

Mechanical hands have application in industries in some of the following aspects:
safety in dangerous workplaces, meat packing
gripping capabilities better than traditional, handle delicate objects,
dampening qualities make it better suited to handle fragile objects.
In extreme heat
Handle chemicals
Hand extensions
Stores soap as cleaning hands
Novelty item

Package

One embodiment of the present invention is a package. Coil Bodies' hollow space is used for transport. Modifiable Coil Body shape is used to accommodate various loads. A package will retain a compact shape by compressing itself over its contents. High strength to weight ratio provides strength without significant weight gain ideal for shipping. The package will demonstrate the usefulness of the hollow body, malleable shape, weight to strength ratio, Coil Joints' ability to expand and contract.

The Package is a Flexible Container
The length of the coil changes
The height of the coil changes
Cone shapes possible
The joint springs compress over the contents
Uses struts to stabilize the shape

The package is stackable and can be stackable.

The package can be used as a strut to secure loads.

The Package with a Double Coil Wall

Increased shock protection for contents and an insulation wall

The Package is Useful After the Delivery

After the package has been delivered to the user, it can be used to collect items to be shipped elsewhere.

The package is useful for the collection of cardboard, and other packaging material to return it to the manufacturer.

The package is useful for merchandise display, or a liquid dispenser.

The package can record and transmit activity with onboard devices.

The package outer shell is changeable

The package has a message space, static or dynamic used to inform or advertise.

The Package will be Strong and Light Weight

Strength characteristics of the coil will be useful in a shipping scenario Tube shape minimizes week corner spots.

### DESCRIPTION OF THE FIGURES

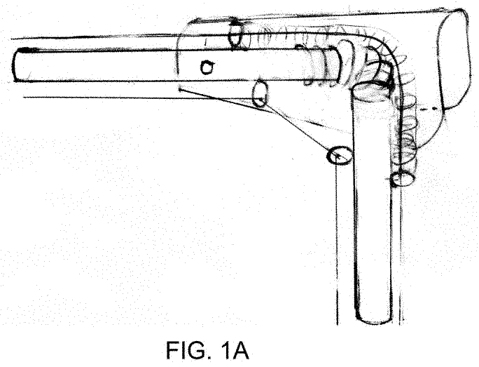

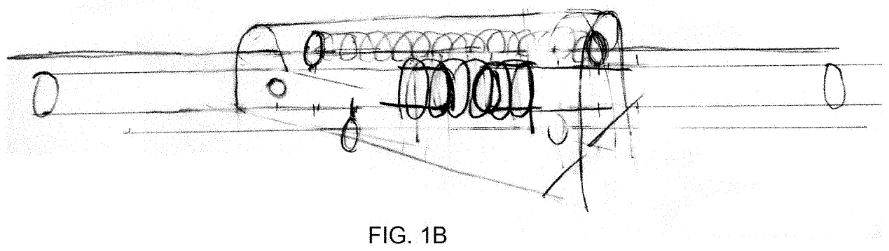

FIG. 1A shows two details of the hand join of one embodiment of the Rod and Coil Construction System.

FIG. 1B shows two details of the hand joint of one embodiment of the Rod and Coil Construction System.

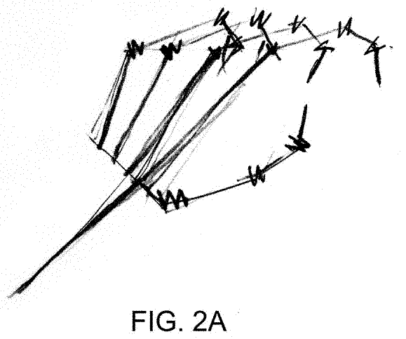

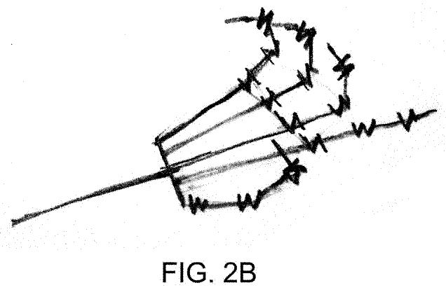

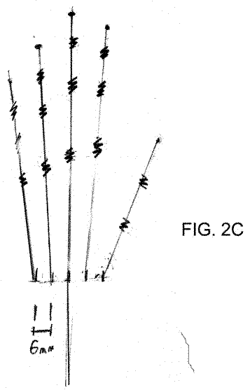

FIG. 2A shows a layout of the joints and how they would be incorporated into the hand of one embodiment of the Rod and Coil Construction System.

FIG. 2B shows a layout of the joints and how they would be incorporated into the hand of one embodiment of the Rod and Coil Construction System.

FIG. 2C shows a layout of the joints and how they would be incorporated into the hand of one embodiment of the Rod and Coil Construction System.

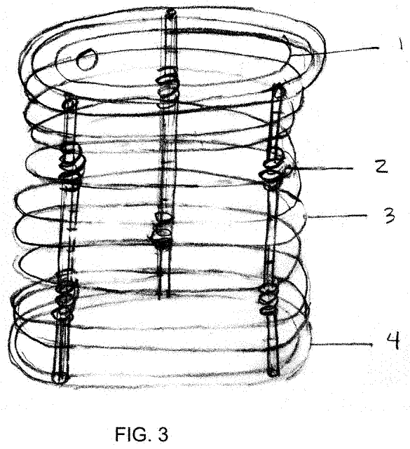

FIG. 3 shows a general image of the package of one embodiment of the Rod and Coil Construction System.

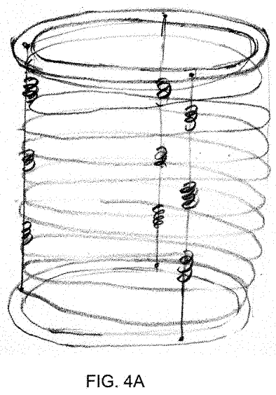

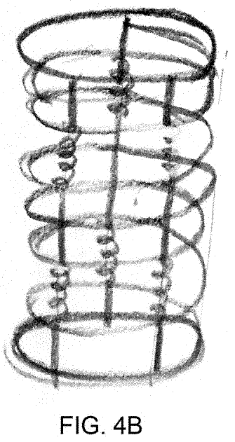

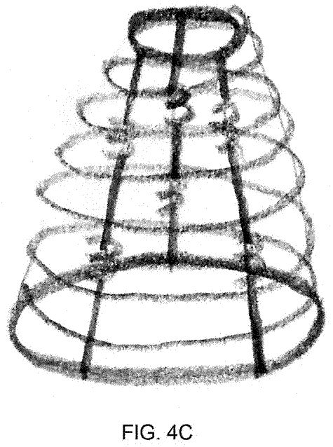

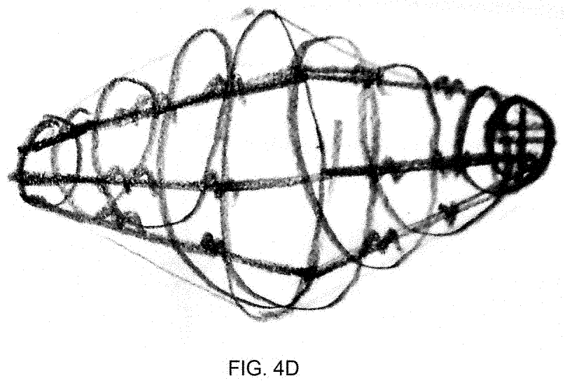

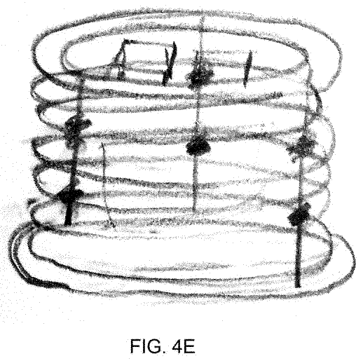

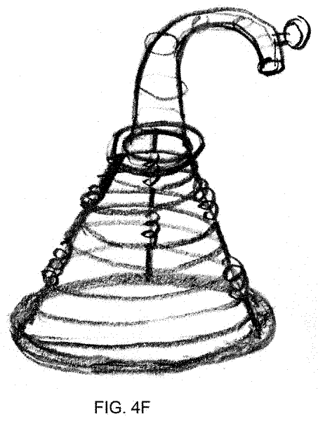

FIG. 4A shapes the package can take and two after delivery uses depicted of one embodiment of the Rod and Coil Construction System.

FIG. 4B shapes the package can take and two after delivery uses depicted of one embodiment of the Rod and Coil Construction System.

FIG. 4C shapes the package can take and two after delivery uses depicted of one embodiment of the Rod and Coil Construction System.

FIG. 4D shapes the package can take and two after delivery uses depicted of one embodiment of the Rod and Coil Construction System.

FIG. 4E shapes the package can take and two after delivery uses depicted of one embodiment of the Rod and Coil Construction System.

FIG. 4F shapes the package can take and two after delivery uses depicted of one embodiment of the Rod and Coil Construction System.

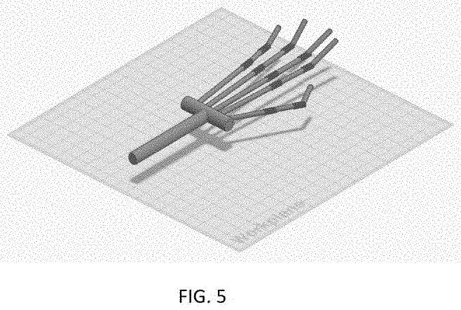

FIG. 5 displays the artificial hand made through the rod and coil construction system

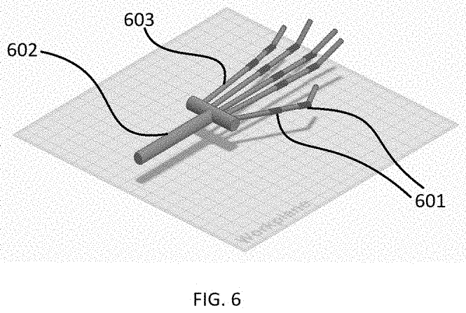

FIG. 6 displays the artificial hand made through the rod and coil construction system, with additional details. The Coil Joint 601 functions as the human joint. The thick rod 602 and thin rod 603 function as the equivalent to human bone.

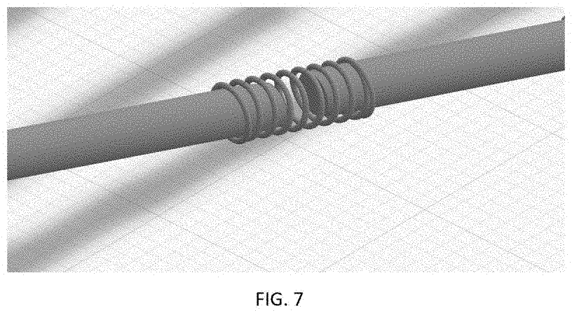

FIG. 7 displays 1 connection in the artificial hand made through the rod and coil construction system

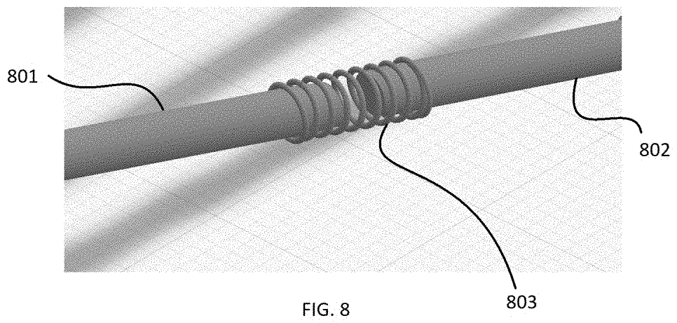

FIG. 8 displays 1 connection in the artificial hand made through the rod and coil construction system, with additional details. The first rod 801 and second rod 802 are connected by a coil 803, that allows for extension and change in direction, which is necessary for flexibility in the design of a package, or of an artificial hand, or any other use of the rod and coil construction system.

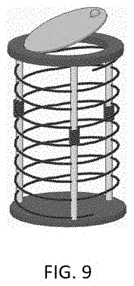

FIG. 9 displays a cylindrical design, in which a coil goes through rods from the bottom to top of an otherwise open-sided cylinder, which has a removable top.

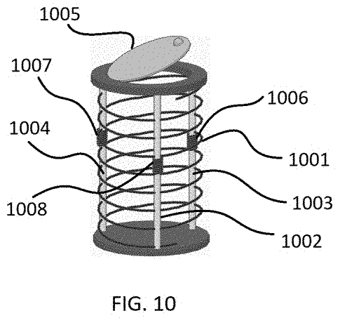

FIG. 10 displays a cylindrical design, in which a coil 1001 goes through rods 1002, 1003, and 1004 from the bottom to top of an otherwise open-sided cylinder, which has a removable top 1005. This version differs from FIG. 9 because it has additional details.

Coil 1001 is a coil body, which is a coil is threaded through one or more rods while retaining its shape, but reinforced by these rods.

There are also 3 coil joints 1006, 1007 and 1008.

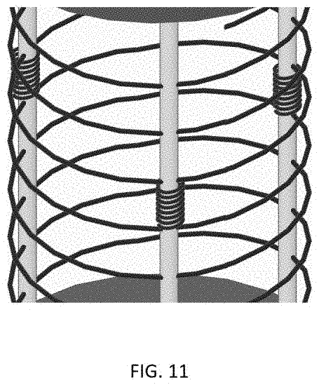

FIG. 11 displays a cylindrical design, in which a coil goes through rods from the bottom to top of an otherwise open-sided cylinder, and at a certain height there are coil joint spines used to secure the connection to the rods, as opposed to the coil going through the rods, which it does everywhere else vertically on the rod.

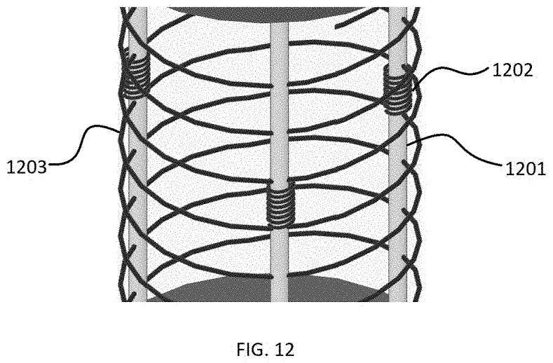

FIG. 12 displays a cylindrical design, in which a coil goes around rods from the bottom to top of an otherwise open-sided cylinder, and at a certain height there are coil joint spines used to secure the connection to the rods, as opposed to the coil going through the rods, which it does everywhere else vertically on the rod. This package differs from FIG. 11 because it contains more details.

Rod 1201 is 1 of 3 rods, with coil body 1203 going around and through them.

There are also 3 coil joint spines such as coil joint spine 1202.

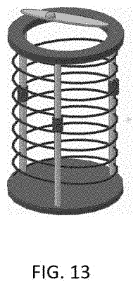

FIG. 13 displays a cylindrical design, in which a coil goes through rods from the bottom to top of an otherwise open-sided cylinder, which has a removable top. At a certain height there are coil joint spines used to secure the connection to the rods, as opposed to the coil going through the rods, which it does everywhere else vertically on the rod. There is also an openable top of the lid.

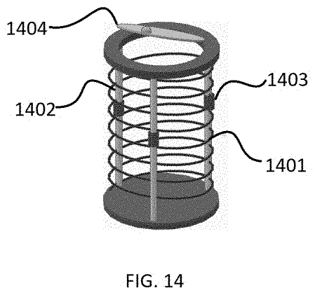

FIG. 14 displays a cylindrical design, in which a coil goes around rods from the bottom to top of an otherwise open-sided cylinder, which has a removable top. At a certain height there are coil joint spines used to secure the connection to the rods, as opposed to the coil going through the rods, which it does everywhere else vertically on the rod. This package differs from FIG. 13 because it contains more details.

Rod 1402 is 1 of 3 rods, with coil body 1401 going around and through them.

There are also 3 coil joint spines such as coil joint spine 1403. There is also an openable top 1404 of the lid.

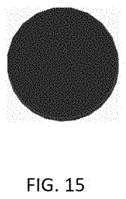

FIG. 15 displays the flat almost circular bottom of the package.

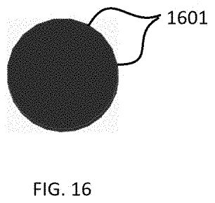

FIG. 16 displays the flat almost circular bottom of the package, with additional details.

The points 1601 are where rods might end or begin at the bottom, with straight lines between points on the bottom.

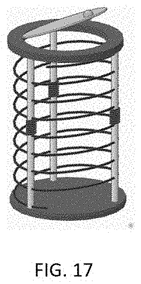

FIG. 17 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod.

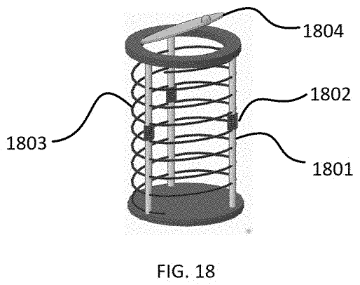

FIG. 18 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod. This package differs from FIG. 17 because it contains more details.

Rod 1801 is 1 of 3 rods, with coil body 1803 going around and through them. There are also 3 coil joints such as coil joint 1802. These coil joints allow the package to expand vertically as well as horizontally, to provide additional flexibility of the package, so that the contents inside the package remain undamaged. There is also an openable top 1804 of the lid.

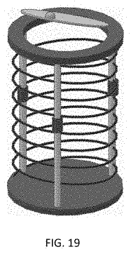

FIG. 19 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod.

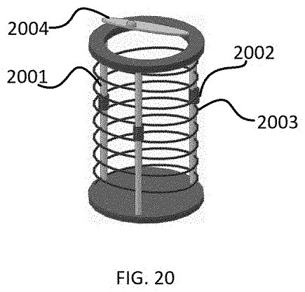

FIG. 20 displays a cylindrical design, in which a coil goes around rods from the bottom to top of an otherwise open-sided cylinder, which has a removable top. At a certain height there are coil joints used to secure the connection to the rods, as opposed to the coil going through the rods, which it does everywhere else vertically on the rod. This package differs from FIG. 19 because it contains more details.

Rod 2001 is 1 of 3 rods, with coil body 2003 going around and through them. There are also 3 coil joints such as coil joint 2002. These coil joints allow the package to expand vertically as well as horizontally, to provide additional flexibility of the package, so that the contents inside the package remain undamaged. There is also an openable top 2004 of the lid.

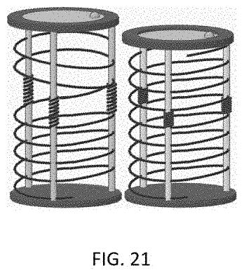

FIG. 21 displays the 2 stages of the package, one in which the package is expanded, and the other in which the package is contracted. There is a total height difference, as well as a reduction in distance between the height of each round of coil, as well as a reduction in the height of the coil joints.

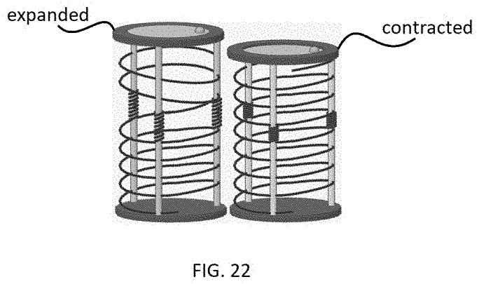

FIG. 22 displays the 2 stages of the package, one in which the package is expanded, and the other in which the package is contracted. There is a total height difference, as well as a reduction in distance between the height of each round of coil, as well as a reduction in the height of the coil joints. This figure differs from FIG. 21 because it has additional details.

The package shown as expanded has coil joints that have expanded, as well as a coil body that has stretched and expanded. This allows the package additional flexibility, for both larger items and so as to not damage the contents inside the damage by being too rigid so as to be damaged if pulled or pushed with too much pressure.

The package shown contracted is the starting point for the coil joints and coil body, and from that point is able to expand to the maximum size shown in the expanded version.

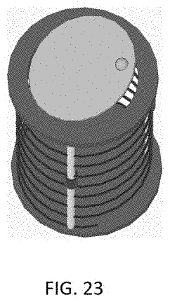

FIG. 23 displays the package from a top viewpoint, and with a full cover, such that there is no opening between the coils or the rods to the outside.

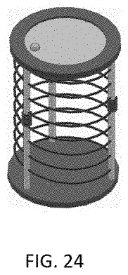

FIG. 24 displays the package from a top viewpoint, and with a full cover, such that there is no opening between the coils or the rods to the outside.

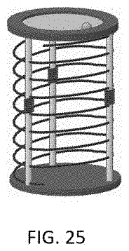

FIG. 25 displays a cylindrical design, in which a coil goes through rods from the bottom to top of an otherwise open-sided cylinder, which has a removable top, and in this case the top is closed. The view is from about 45 degrees up, between horizontal and vertical.

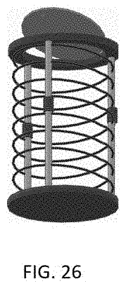

FIG. 26 displays a cylindrical design, in which a coil goes through rods from the bottom to top of an otherwise open-sided cylinder, which has a removable top, and in this case the top is closed. The view is from about 45 degrees up, between horizontal and vertical.

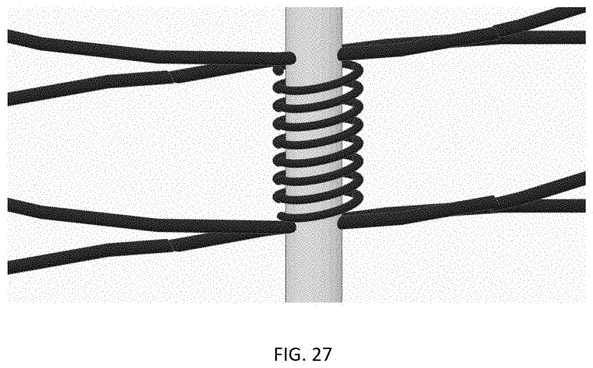

FIG. 27 displays a cylindrical design, in which a coil goes through rods from the bottom to top of an otherwise open-sided cylinder, which has a removable top, and in this case the top is closed. The view is almost horizontal.

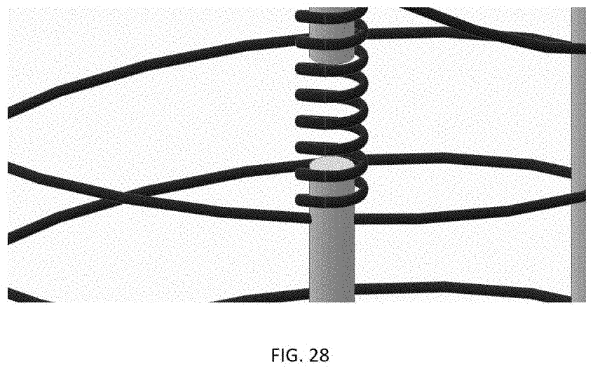

FIG. 28 displays a cylindrical design, in which a coil goes through rods from the bottom to top of an otherwise open-sided cylinder, which has a removable top, and in this case the top is closed. The view is almost horizontal. This figure differs from FIG. 27 because it has additional details.

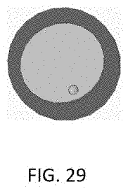

FIG. 29 displays a cylindrical design, in which a coil goes through rods from the bottom to top of an otherwise open-sided cylinder, which has a removable top, and in this case the top is closed. The view is from below the horizontal plane.

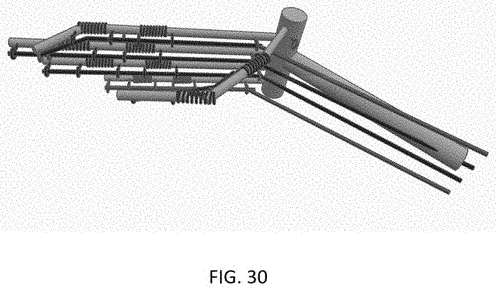

FIG. 30 displays an artificial hand with rods and coil joints.

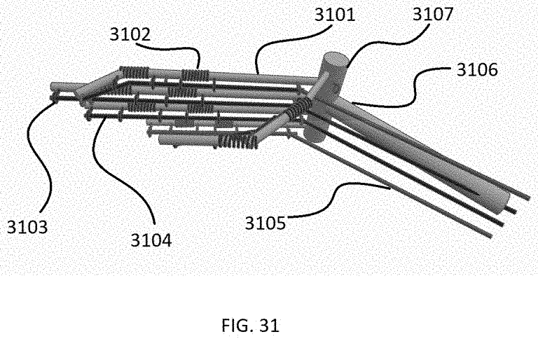

FIG. 31 displays an artificial hand with rods and coil joints. This package differs from FIG. 30 because it contains more details.

Rod 3106 is thick and simulates the forearm of the hand. Horizontal rod 3017 simulates the wrist, and provides rotational capability as it rotates on Rod 3107. Five rods 310 simulate fingers, while multiple coil joints 3102 simulate knuckles, in the sense that they can bend.

Five rods 3105 represent thinner rods that move in tune with the movement of the fingers of the artificial hand. This is done by exerting pull on additional five thin rods 3104, which are connected to the five rods 3101 by loops 3103. As such, when pull is exerted on five rods 3105, then that pull is exerted on additional five thin rods 3104, which pulls loops 3103, which causes movement in five rods 3101 and multiple coil joints 3102. This series of movements thus simulates the movement of a hand with fingers and knuckles. It can be modified to focus on 1 or more fingers, as well as 1 or more knuckles, allowing for highly detailed and coordinated movement by the artificial hand.

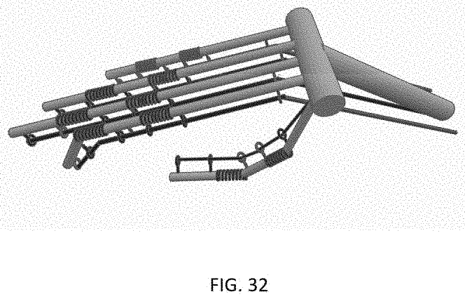

FIG. 32 displays an artificial hand with rods and coil joints.

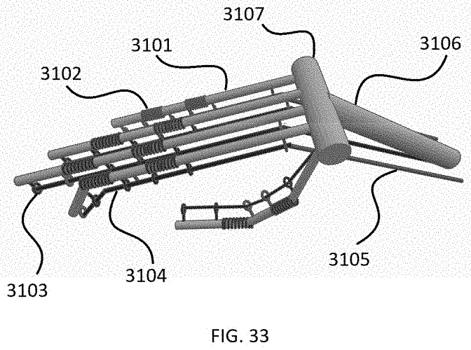

FIG. 33 displays an artificial hand with rods and coil joints. This package differs from FIG. 32 because it contains more details.

Rod 3106 is thick and simulates the forearm of the hand. Horizontal rod 3017 simulates the wrist, and provides rotational capability as it rotates on Rod 3107. Five rods 3101 simulate fingers, while multiple coil joints 3102 simulate knuckles, in the sense that they can bend.

Five rods 3105 represent thinner rods that move in tune with the movement of the fingers of the artificial hand. This is done by exerting pull on additional five thin rods 3104, which are connected to the five rods 3101 by loops 3103. As such, when pull is exerted on five rods 3105, then that pull is exerted on additional five thin rods 3104, which pulls loops 3103, which causes movement in five rods 3101 and multiple coil joints 3102. This series of movements thus simulates the movement of a hand with fingers and knuckles. It can be modified to focus on 1 or more fingers, as well as 1 or more knuckles, allowing for highly detailed and coordinated movement by the artificial hand.

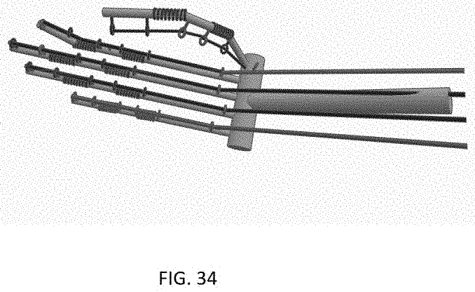

FIG. 34 displays an artificial hand with rods and coil joints.

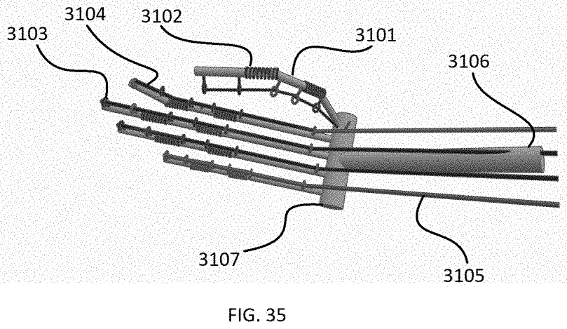

FIG. 35 displays an artificial hand with rods and coil joints. This package differs from FIG. 34 because it contains more details.

Rod 3106 is thick and simulates the forearm of the hand. Horizontal rod 3017 simulates the wrist, and provides rotational capability as it rotates on Rod 3107. Five rods 3101 simulate fingers, while multiple coil joints 3102 simulate knuckles, in the sense that they can bend.

Five rods 3105 represent thinner rods that move in tune with the movement of the fingers of the artificial hand. This is done by exerting pull on additional five thin rods 3104, which are connected to the five rods 3101 by loops 3103. As such, when pull is exerted on five rods 3105, then that pull is exerted on additional five thin rods 3104, which pulls loops 3103, which causes movement in five rods 3101 and multiple coil joints 3102. This series of movements thus simulates the movement of a hand with fingers and knuckles. It can be modified to focus on 1 or more fingers, as well as 1 or more knuckles, allowing for highly detailed and coordinated movement by the artificial hand.

### Additional Embodiments

Another embodiment of the present invention is a system of construction based on rods and coils, that can be utilized to make any of the following:

a coil joint, which is a mechanism that connects two solid bodies, shaped in a helix structure;
a coil body, which is a helix threaded through one or more reinforcing rods, and has the capability of holding contents; a coil package, which is a container for protecting, securing and transporting objects of various sizes shape and quantity; coil action walls, wherein the coils expand and contract to contain and secure contained objects in place; wherein the coil shape provides a high strength to weight ratio.

This embodiment can be further refined by adding a coil rod spine, which is a series of coil joints connected by rods to build a flexible yet robust structure.

This embodiment can also be refined such that the coil joints have the capability of 360-degree rotation; the coil joints also have the capability to extend; and the coil joints also have the capability to contract.

Another embodiment of the present invention is an apparatus of a package that is made of a coil body, reinforced by rods, where the coil is threaded through a rod, and the coil retains its shape but is reinforced by the rod; wherein the package has a high strength to weight ratio; wherein the package is collapsible, stretchable, and malleable; wherein the package has a hollow body; wherein the package has a high dimensional stability; and wherein the package retains a compact shape by compressing itself over its contents.

This embodiment may be refined such that the shape of the package is a helix shape.

This embodiment may also be refined by adding a coil rod spine, which is a series of coil joints connected by rods to build a flexible yet robust structure, and wherein the coil joints have the capability of 360-degree rotation.

This embodiment may also be refined by adding coil action walls, wherein the coils expand and contract, allowing the package to expand and contract; wherein this expanding and contracting of the package improves the containment of any objects contained in the package, and further secures the objects contained in the package. This embodiment may be further refined by adding 2 coil action walls surrounding the package, which results in increased shock protection for the objects in the package, and results in additional insulation of the objects in the package against the environment outside of the package.

Another embodiment of the present invention is An apparatus that is an artificial hand, capable of grasping and individual finger movements by utilizing coil joints that simulate human joints and a coil body that simulates the human shape of the hand, and contains a hollow space, comprising: a coil joint, which is a mechanism that connects two solid bodies, shaped in a helix structure; a coil body, which is a helix threaded through one or more reinforcing rods, and has the capability of holding contents.

This embodiment may be further refined by adding spring joints that function as the equivalent to human joints, rods that function as the equivalent to human bones, wherein the spring joints create a dampening effect that simulates the dampening effect in human hands, wherein the spring joints have fill rotational motion, extending motion, and contracting motion.

This embodiment may be further refined such that the internal space of the coil body is filled with actuators, wherein the actuators can be used to move and control the artificial hand, and wherein the actuators are powered by electricity.

Another embodiment of the present invention is a system of construction based on rods and coils, that comprises a coil joint, a coil body, a coil spine and one or more apparatuses. There can be multiple combinations of any and all of these, such that the result is a variety of distinct structures that have improved characteristics compared to their predecessors.

One technique used for the construction of the apparatus is a coil joint, which has 360 degree rotation, wherein the rotation can be limited to simulate other types of joints. This coil joint can be combined with other joint styles to create hybrid styles.

The embodiment has 2 bias actions, one is an extending action and the other is a contracting action.

Another aspect of the embodiment is that the action of the coil results in a more life-like motion than not using the coil.

Another embodiment of the present invention is the construction of a coil body, which has a flexible shape that can be locked into place. The coil body has a high strength to weight ratio, which leads to high dimensional stability. There is also hollow space for storage and transport of a package inside of the coil body.

Another embodiment of the present invention is the coil spine, which simulates the spines of living beings. It is a flexible yet robust structure.

Another embodiment of the present invention is the package, which is a coil body made with coil spines. The compression action of the spring compresses over the contents within the package. The package has a flexible shape due to the coil body made with coil spines. The flexible shape can be locked into place. The coils have vibrational qualities. The package can form part of a shock absorber system. Alternatively, the package can form part of a strut system. Furthermore, the coil body can have double walls for extra protection of contents.

Another embodiment of the present invention is a mechanical hand. The coil joints that function as the equivalent to human joints. There are also rods that function as the equivalent to human bones. The coil joints that create a dampening that simulates the dampening effect in human hands. There are also coil bodies of the hand, that are used for transport and storage. The fingers of the artificial hand can be replaced with tools that are of a similar size and not excessive weight. The coil joints can be modified to simulate various human joints. The bias of the coil joints increases the ability of the artificial hand to grasp. The bias of the coil joint functions as tendon tension. The bias of all the coils results in more life-like motion. Movement of the finger can be performed with a single wire opposing the bias of the coil. Optionally, this embodiment can have autonomous motion by utilizing an actuator system.

Another embodiment of the present invention is a method of construction based on rods and coils, that includes multiple parts comprising: distinct apparatuses, one or more coil joints, a coil body, and one or more coil spines; wherein any combination of the above parts can result in a variety of distinct structures; the coil joint has 360 degree rotation; the rotation can be limited to simulate other types of joints; this coil joint can be combined with other joint styles to create hybrid styles; there are 2 bias actions, one is an extending action and the other is a contracting action; the action of the coil results in a more life-like motion than not using the coil.

This embodiment may be further refined, such that the coil body has a flexible shape that can be locked into place; the coil body has a high strength to weight ratio, which leads to high dimensional stability; there is also hollow space for storage and transport of a package inside of the coil body.

The embodiment may be alternatively refined such that it further comprises the coil spine simulates the spines of living beings; wherein the coil spine is a flexible yet robust structure.

The embodiment may be alternatively refined such that it further comprises: a package, which is a coil body made with coil spines; wherein the compression action of the spring compresses over the contents within the package; wherein the package has a flexible shape due to the coil body made with coil spines; wherein the flexible shape can be locked into place; wherein all coils have vibrational qualities.

Another embodiment of the present invention is an apparatus of a mechanical hand based on rods and coils, that includes multiple parts comprising: one or more coil joints, a coil body, and one or more coil spines; wherein the coil joints function as the equivalent to human joints; there are rods that function as the equivalent to human bones; the coil joints create a dampening effect that simulates the dampening effect in human hands; there are coil bodies of the mechanical hand, that are used for transport and storage.

The embodiment may be alternatively refined such that it further comprises' the fingers of the artificial hand can be replaced with tools that are of a similar size and not excessive weight.

The embodiment may be alternatively refined such that it further comprises: the coil joints can be modified to simulate various human joints; where there is a bias in all coils and coil joints; the bias of the coil joints increases the ability of the artificial hand to grasp; the bias of the coil joint functions as tendon tension; the bias of all the coils results in more life-like motion; movement of the finger can be performed with a single wire opposing the bias of the coil.

The embodiment may be alternatively refined such that it further comprises: the artificial hand can have autonomous motion by utilizing an actuator system.

Having described the invention by the description and illustrations above, it should be understood that these are exemplary of the invention and are not to be considered as limiting. Accordingly, the invention is not to be considered as limited by the foregoing description, but includes arty equivalents.

What is claimed is:

1. A system of construction based on rods and coils, that includes multiple parts comprising:
distinct apparatuses, one or more coil joints, a coil body, and one or more coil spines;
wherein any combination of the above parts can result in a variety of distinct structures.

2. The system of claim 1, further comprising:
wherein the coil joint has 360 degree rotation;
wherein the rotation can be limited to simulate other types of joints;
wherein this coil joint can be combined with other joint styles to create hybrid styles;
wherein there are 2 bias actions, one is an extending action and the other is a contracting action;
wherein the action of the coil results in a more life-like motion than not using the coil.

3. The system of claim 1, further comprising:
wherein the coil body has a flexible shape that can be locked into place;
wherein the coil body has a high strength to weight ratio, which leads to high dimensional stability;
wherein there is also hollow space for storage and transport of a package inside of the coil body.

4. The system of claim 1, further comprising:
wherein the coil spine simulates the spines of living beings;
wherein the coil spine is a flexible yet robust structure.

5. The system of claim 1, further comprising:
a package, which is a coil body made with coil spines;
wherein the compression action of the spring compresses over the contents within the package;
wherein the package has a flexible shape due to the coil body made with coil spines;
wherein the flexible shape can be locked into place;
wherein all coils have vibrational qualities.

6. The system of claim 5, further comprising:
wherein the package forms part of a shock absorber system.

7. The system of claim 5, further comprising:
wherein the package forms part of a strut system.

8. The system of claim 5, further comprising:
wherein the coil body can have double walls for extra protection of contents.

9. The system of claim 1, further comprising,
a mechanical hand;
wherein the coil joints function as the equivalent to human joints;
wherein there are rods that function as the equivalent to human bones;
wherein the coil joints create a dampening effect that simulates the dampening effect in human hands;
wherein there are coil bodies of the hand, that are used for transport and storage.

10. The system of claim 9, further comprising:
wherein the fingers of the artificial hand can be replaced with tools that are of a similar size and not excessive weight.

11. The system of claim 9, further comprising:
wherein the coil joints can be modified to simulate various human joints;
where there is a bias in all coils and coil joints;
wherein the bias of the coil joints increases the ability of the artificial hand to grasp;
wherein the bias of the coil joint functions as tendon tension;
wherein the bias of all the coils results in more life-like motion;
wherein movement of the finger can be performed with a single wire opposing the bias of the coil.

12. The system of claim 9, further comprising:
wherein the artificial hand can have autonomous motion by utilizing an actuator system.

13. A method of construction based on rods and coils, that includes multiple parts comprising: distinct apparatuses, one or more coil joints, a coil body, and one or more coil spines;
wherein any combination of the above parts can result in a variety of distinct structures;
wherein the coil joint has 360 degree rotation;
wherein the rotation can be limited to simulate other types of joints;
wherein this coil joint can be combined with other joint styles to create hybrid styles;
wherein there are 2 bias actions, one is an extending action and the other is a contracting action;
wherein the action of the coil results in a more life-like motion than not using the coil.

14. The method of claim 13, further comprising:
wherein the coil body has a flexible shape that can be locked into place;
wherein the coil body has a high strength to weight ratio, which leads to high dimensional stability;
wherein there is also hollow space for storage and transport of a package inside of the coil body.

15. The method of claim 13, further comprising:
wherein the coil spine simulates the spines of living beings;
wherein the coil spine is a flexible yet robust structure.

16. The method of claim 13, further comprising:
a package, which is a coil body made with coil spines;
wherein the compression action of the spring compresses over the contents within the package;
wherein the package has a flexible shape due to the coil body made with coil spines;
wherein the flexible shape can be locked into place;
wherein all coils have vibrational qualities.

17. An apparatus of a mechanical hand based on rods and coils, that includes multiple parts comprising:
one or more coil joints, a coil body, and one or more coil spines;
wherein the coil joints function as the equivalent to human joints;
wherein there are rods that function as the equivalent to human bones;
wherein the coil joints create a dampening effect that simulates the dampening effect in human hands;
wherein there are coil bodies of the mechanical hand, that are used for transport and storage.

18. The apparatus of claim 17, further comprising:
wherein the fingers of the artificial hand can be replaced with tools that are of a similar size and not excessive weight.

19. The apparatus of claim 17, further comprising:
wherein the coil joints can be modified to simulate various human joints;
where there is a bias in all coils and coil joints;
wherein the bias of the coil joints increases the ability of the artificial hand to grasp;
wherein the bias of the coil joint functions as tendon tension;
wherein the bias of all the coils results in more life-like motion;
wherein movement of the finger can be performed with a single wire opposing the bias of the coil.

20. The apparatus of claim 17, further comprising:
wherein the artificial hand can have autonomous motion by utilizing an actuator system.
